(19) United States
(12) Patent Application Publication
(10) Pub. No.: US 2026/0256974 A1
(43) Pub. Date: Sep. 3, 2026

(54) BOOSTER DEVICE FOR AROMA DISPLAY

(71) Applicant: AROMAJOIN CORPORATION, Kyoto-shi (JP)

(72) Inventors: Dong Wook KIM, Kyoto-shi (JP); Aiko YASUI, Kyoto-shi (JP)

(21) Appl. No.: 18/865,058

(22) PCT Filed: Jun. 15, 2023
(86) PCT No.: PCT/JP2023/022229

§ 371 Date: Nov. 12, 2024

(30) Foreign Application Priority Data
Jul. 13, 2022 (JP) 2022-112399

(51) Int. Cl. A61L 9/14

(52) U.S. Cl. CPC A61L 9/14; A61L 2209/15

(57) ABSTRACT

A booster device for an aroma display, which is compact even when combined with the aroma display and is capable of delivering aroma far away is provided. The booster device for an aroma display is a booster for enhancing aroma emission power of an aroma display 60 that has upper and lower surfaces and a side surface connecting outer circumferences of the upper and lower surfaces and emitting aroma from the upper surface. The booster device has a holder part 62 for holding the aroma display, a duct 130 having a first opening and a second opening 66 provided at the holder part 62 along the side surface of aroma display, and a fan to feed air to the first opening of duct 130. The first opening is formed on the side of bottom surface of the aroma display held by holder part 62, and the second opening 66 is formed on the side of upper surface of the aroma display.

### TECHNICAL FIELD

The present invention relates to an aroma display that emits scented air and, more specifically, to a booster device for extending reach of the air from the aroma display. The present application claims convention priority on Japanese patent application No. 2022-112399 filed on Jul. 13, 2022, the entire contents of which is incorporated herein by reference.

### BACKGROUND ART

Recently, devices have been proposed which are used with audio-visual reproducing devices such as television receivers, personal computers and game machines for generating scents fitting for the time and place. In the present Specification, such a device that generates scents fitting for scenes will be referred to as an “aroma display”. Particularly, if aroma is used in virtual reality, it is found that not only the scent of things is felt by the user but the sense of speed, for example, can be felt by the brain function induced by olfactory sensation.

In order to generate aroma appropriately scene by scene, it is necessary to timely switch scents. Therefore, an aroma display is desirable that can diffuse a desired scent in a certain limited scope at one time point and can blow out that scent and switch to another scent at another time point. On the other hand, it is assumed that an aroma display is used by an individual user to feel aroma or flagrance.

Therefore, it is desirable that the aroma display has a small size and that the sound of aroma emission is low. In an aroma display as such, it is difficult to blow out a jet of air with such a force that is sufficient to dissipate any lingering scent immediately.

In order to cope with such a problem, it may be possible to increase the power of aroma emitting mechanism. Increase of the aroma emitting power, however, inevitably involves increase in size of the aroma display and increase of noise at the time of operation.

Patent Literature 1 specified below proposes a solution to such a problem.

Patent Literature 1 proposes a booster device that can blow out a jet of air with larger power than the aroma display, rather than increasing the power of emission of aroma display. The booster device accommodates an aroma display in its housing, and using an axial fan (propeller fan) provided in the booster device, generates an air flow around the aroma display. The aroma display can be removed at any time from the booster device. Therefore, if it is unnecessary to extend the reach of aroma, the aroma display may be used on its own, and if it is necessary to diffuse aroma widely or to immediately dissipate any scent, the aroma display may be used combined with the booster device.

The booster device disclosed in Patent Literature 1 attains the effect that aroma can reach further by larger air flow (flow rate) for an aroma display that can emit aroma with only a small power on its own.

### CITATION LIST

### Patent Literature

PTL 1: JP2020-162644A

### SUMMARY OF INVENTION

### Technical Problem

The booster device disclosed in Patent Literature 1, however, has a problem that it is large. Therefore, when the aroma display is to be used personally, the booster device must be removed. Further, when air is fed with a greater power by the booster device disclosed in Patent Literature 1, while larger air flow (flow rate) can be attained, the power to push out the air (static pressure) is insufficient due to the characteristic of axial fan. Therefore, aroma emission distance is not as long as expected.

Therefore, an object of the present invention is to provide a booster device for aroma display that is small in size even when combined with the aroma display and that can deliver aroma far away.

### Solution to Problem

According to a first aspect, the present invention provides a booster device for an aroma display having a first surface, a second surface opposite to the first surface, and a third surface connecting outer circumferences of the first and second surfaces and discharging aroma from the first surface; the booster device is for enhancing aroma emission power, and it includes: a holder part for holding the aroma display; a duct having first and second openings, provided along the third surface; and a centrifugal fan for feeding air to the first opening; wherein the duct is provided on the holder part such that the first opening is positioned on the side of the second surface of the aroma display held by the holder part and the second opening is positioned on the side of the first surface of the aroma display held by the holder part.

Preferably, the holder part includes: a display holder part holding that portion of the third surface which is adjacent to the second surface; and a housing part provided at that portion of display holder part which is opposite to the aroma display, for accommodating the centrifugal fan.

More preferably, the holder part and the duct are formed to open at least a part of the third surface of aroma display.

Further preferably, the duct has the same cross-sectional area from the first to the second opening.

Preferably, the duct has the same cross-sectional shape from the first to the second opening.

More preferably, the inner shape of duct is a prism having the first and second openings at opposite ends.

More preferably, the aroma display has a central axis connecting the geometric center of the second surface and the geometric center of the first surface; the outer circumference has a rotational symmetrical shape around the central axis; and the duct is formed to be adjacent to a portion smaller than half the outer circumference of the aroma display in a cross-section perpendicular to the central axis.

Preferably, the outer circumference of aroma display is a polygon around the central axis; and the duct is formed to be adjacent to only those parts of the polygon that are less than N/2 continuous sides, where N is the number of corners of the polygon.

More preferably, the polygon is a regular polygon.

More preferably, the regular polygon is a regular hexagon, and the duct is formed to be adjacent only to those parts of the hexagon that are less than two continuous sides.

Preferably, the duct is formed to be adjacent only to one side of the regular hexagon.

More preferably, the aroma display has a central axis connecting the geometrical center of the second surface and the geometrical center of the first surface; the centrifugal fan is mounted on the holder part such that its air outlet faces a direction intersecting the central axis, or a direction not intersecting or parallel to the central axis; and the aroma display further includes a deflector duct, which is connected to the air outlet of centrifugal fan and the duct such that the air fed out from the air outlet of centrifugal fan is guided through the first opening to the duct.

More preferably, the booster device includes one or more flow straighteners provided in the duct, formed along the direction from the first to the second openings.

Preferably, the centrifugal fan is mounted on the holder part such that the rotation axis of its impeller is parallel to the central axis.

More preferably, the centrifugal fan is mounted on the holder part such that the rotation axis of its impeller faces the direction intersecting the central axis, or the direction not intersecting or parallel to the central direction.

Further preferably, on the third surface of aroma display, a female screw part is formed; on the holder part, a positioning member is formed to define the position of aroma display such that the female screw position has a prescribed relation with the holder when the aroma display is held; the holder part has a sidewall formed at an area surrounding the position facing the female screw when the aroma display is held; and at that position of the sidewall which faces the female screw, an opening of a diameter larger than the nominal diameter of female screw is formed.

Preferably, the booster device further includes: a stand, engaging with the female screw part formed on the third surface of aroma display through the opening on the sidewall of holder part and thereby supporting the aroma display and the booster device locked with each other.

More preferably, the stand has a base part defining upper and lower surfaces; and a support part mounted on the base part to be perpendicular to the upper surface of the base part and having an opening through which a locking screw to be engaged with the female screw passes, and on the lower surface of the base part, one or more magnets are mounted.

The outer circumference of aroma display has a polygonal shape, and the duct is formed to be adjacent only to those parts of the polygon that are less than two continuous sides.

The booster device further includes a stand that engages with the engaging section/part formed on the third surface of aroma display through an opening formed in the sidewall of the holder part, for swingably holding the aroma display and the booster device.

The foregoing and other objects, features, aspects and advantages of the present invention will become more apparent from the following detailed description of the present invention when taken in conjunction with the accompanying drawings.

### BRIEF DESCRIPTION OF DRAWINGS

FIG. 1 is a perspective view showing an overall structure of an aroma display system using the booster device for aroma display in accordance with a first embodiment of the present invention.

FIG. 2 is an exploded perspective view of the aroma display system shown in FIG. 1.

FIG. 3 shows a state of use of the aroma display system shown in FIG. 1.

FIG. 4 is a perspective view of the aroma display from above.

FIG. 5 is a perspective view of the aroma display from the bottom.

FIG. 6 is a perspective view of the booster as the boosting device in accordance with the first embodiment of the present invention.

FIG. 7 is a plan view of the booster holder shown in FIG. 6.

FIG. 8 is a bottom view of the booster holder shown in FIG. 6.

FIG. 9 is an enlarged plan view showing portions near the upper side opening of the duct shown in FIG. 7.

FIG. 10 is a cross-sectional view of the holder along the line 10-10 of FIG. 7.

FIG. 11 is a cross-sectional view of the holder along the line 11-11 of FIG. 7.

FIG. 12 is a perspective view of the deflector duct viewed from the front side.

FIG. 13 is a perspective view of the deflector duct viewed from the rear side.

FIG. 14 is a perspective view of a cross-section of deflector duct cut along 14-14 plane of FIG. 13.

FIG. 15 is a perspective view of the stand of the aroma display system.

FIG. 16 is an exploded perspective view of the aroma display system in accordance with a second embodiment of the present invention.

FIG. 17 is a front view of the aroma display system in accordance with a third embodiment of the present invention.

FIG. 18 is a side view of the aroma display system in accordance with the third embodiment.

FIG. 19 is a perspective view of a fan attachment member with a deflector duct of the aroma display system in accordance with the third embodiment.

FIG. 20 is a perspective view of the fan attachment member cut along 20-20 plane of FIG. 19.

### DESCRIPTION OF EMBODIMENTS

In the following description and in the drawings, the same components are denoted by the same reference characters. Therefore, detailed description thereof will not be repeated.

### 1. First Embodiment

### A. Aroma Display System 50

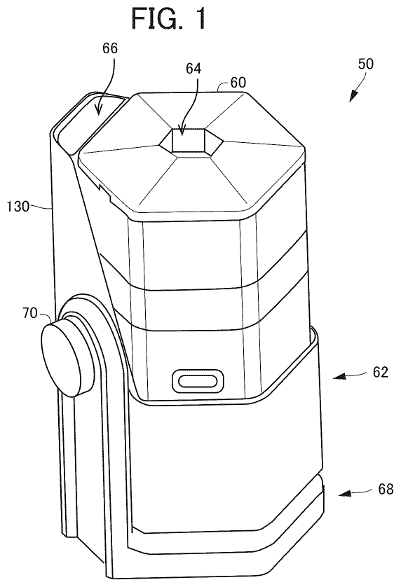

FIG. 1 is a perspective view of an aroma display system 50 in accordance with the first embodiment of the present invention. Referring to FIG. 1, aroma display system 50 includes: an aroma display 60; a booster 62 for holding aroma display 60 and generating an air flow to deliver a jet of air emitted from aroma display 60 over a long distance; and a stand 68 having a booster lock 70 to be engaged with a female screw part (not shown in FIG. 1) formed on aroma display 60 across booster 62, for holding aroma display 60 and booster 62 in a swingable manner around a rotation axis defined by the booster lock 70. The female screw part forms an engaging part for engaging aroma display 60 with booster lock 70 and, in addition, fixes aroma display 60 on a commercially available tripod and the like.

As will be described later, aroma display 60 has upper and bottom surfaces both having the shape of a regular hexagon. At the center of the upper surface of aroma display 60, an opening 64 is formed, from which a jet of scented air is emitted. Booster 62 has a duct 130 that serves as an air passage. Duct 130 is formed along a side surface of aroma display 60. Duct 130 has an opening 66 as an air-emission outlet at a position of substantially the same height as the upper outer circumference of aroma display 60.

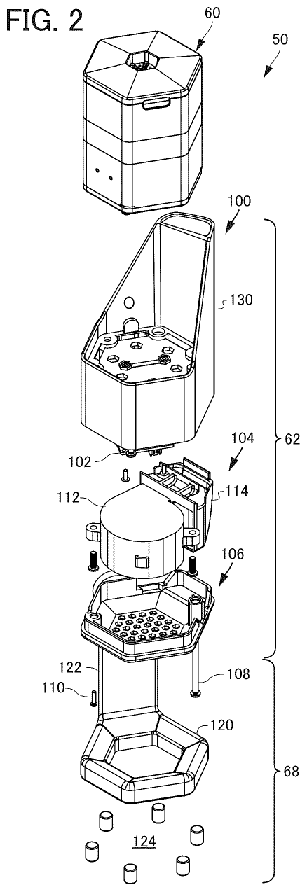

Referring to FIG. 2, booster 62 includes: a holder 100 for holding the bottom surface of aroma display 60 and the outer circumference near the bottom surface of aroma display 60; a substrate 102 attached to the bottom surface of holder 100; and a fan assembly 104 housed in a space formed below holder 100, for feeding air to a bottom side opening (not shown in FIG. 2) of duct 130. Booster 62 further includes a bottom plate 106 engaged to the bottom-side end surface of holder 100.

Substrate 102 has a pin that electrically engages with a connector part formed on the bottom surface of aroma display 60 mounted on holder 100. Substrate 102 is to receive power and control signals from aroma display 60 to fan assembly 104 through this pin. Substrate 102 is screw-fixed on the lower surface of holder 100 by a small screw.

Fan assembly 104 includes: a centrifugal fan 112 for generating the air flow to be fed to the inside of duct 130; and a deflector duct 114 having a first opening fixed at a discharge outlet of centrifugal fan 112 and a second opening coupled to the opening on the bottom surface of duct 130. Centrifugal fan 112 is arranged such that its impeller rotates about an axis parallel to the central axis of aroma display 60. The discharge outlet of centrifugal fan 112 faces a direction not intersecting or parallel to the central axis of centrifugal fan 112. Deflector duct 114 is to turn the air flow, which is blown out from the discharge outlet of centrifugal fan 112 toward the side surface of holder 100, by 90 degrees and to feed the air flow to the inside of duct 130. Centrifugal fan 112 and deflector duct 114 are screw-fixed on the bottom surface of holder 100 by a small screw.

A large number of air inlets are formed for the centrifugal fan 112, on bottom plate 106. Bottom plate 106 is screw-fixed on the bottom surface of holder 100 by a small screw 110. Further, bottom plate 106 and holder 100 have openings that pass from the bottom surface of bottom plate 106 to the upper surface of that portion of holder 100 which houses aroma display 60, when bottom plate 106 is fixed on holder 100. Further, at the same position on the bottom surface of aroma display 60, a female screw part, as an engaging part with stand 68, is formed. By inserting a long screw 108 through this opening from the side of bottom plate 106 to the inside of holder 100 to be engaged with the female screw part at the bottom surface of aroma display 60, bottom plate 106, fan assembly 104, holder 100 and aroma display 60 are fixed as one body.

Stand 68 includes a bottom part 120 that sits stably on a flat plane, of which circumference has a regular hexagonal shape, and a lock holder plate 122 raising perpendicularly from one side of outer circumference of bottom part 120. On lock holder plate 122, a booster lock 70 (see FIG. 1), not shown in FIG. 2, is attached. The structure of lock holder plate 122 will be described later. On the lower surface of bottom part 120, six magnets 124 are embedded. As a result, stand 68 can be attached to a side panel of a steel cabinet and the like and, hence, aroma display system 50 can be placed at various positions.

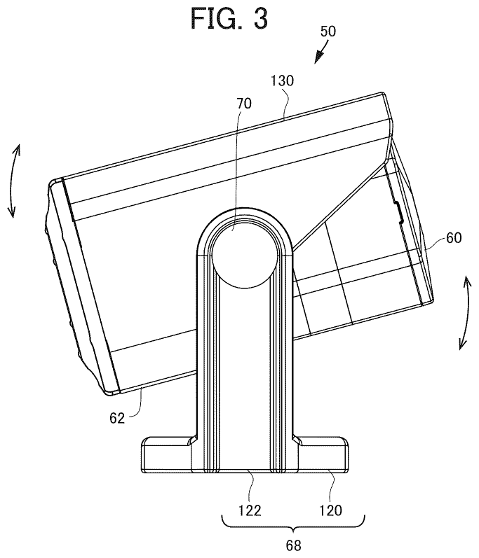

Referring to FIG. 3, an opening, not shown, is formed near the upper end of lock holder plate 122 of stand 68, and booster lock 70 is attached to the opening. As will be described later, booster lock 70 has a male screw that engages with the female screw part provided on the side surface of aroma display 60 across booster 62. By screwing together the male screw part of booster lock 70 and the female screw part of aroma display 60, aroma display 60 and booster 62 are screw-fixed on stand 68. Further, by loosening booster lock 70, it is possible to swing aroma display 60 and booster 62 about the rotation axis of booster lock 70, as indicated by an arrow in FIG. 3. In the present embodiment, the height of booster lock 70 from the bottom part 120 is selected such that when aroma display 60 and booster 62 are mounted, they can pivot only in a prescribed angular range.

### B. Aroma Display 60

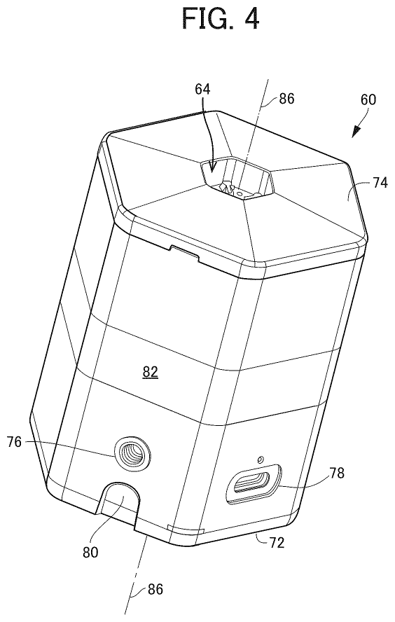

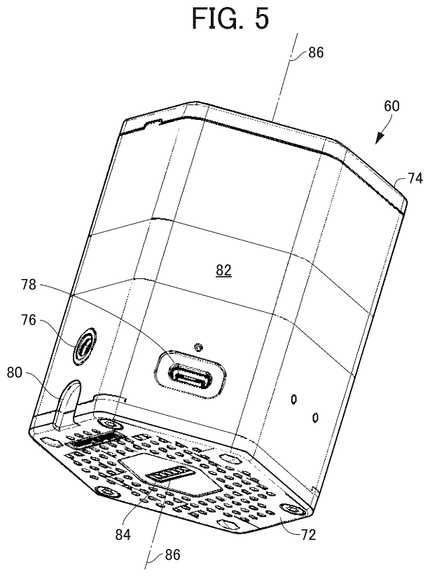

Referring to FIGS. 4 and 5, aroma display 60 has an upper surface 74 as the first surface and a lower surface 72 as the second surface, both having substantially the same regular hexagonal-shaped outer periphery, and a side surface 82 as the third surface connecting the outer peripheries of the lower surface 72 and upper surface 74. Therefore, the shape of aroma display is substantially a regular hexagonal column. Specifically, side surface 82 has six surfaces, that is, from the first to the sixth surfaces. Aroma display 60 has a central axis 86 connecting the geometrical center of hexagon of the lower surface 72 and the geometrical center of hexagon of the upper surface 74. Aroma display 60 has an outer shape of six-order rotational symmetry with the central axis 86 being the axis of rotation.

On the first surface of side surface 82, a female screw part 76 is formed, with which the mail screw part of booster lock 70 engages. At a portion of first surface continuous from the bottom surface, a positioning section 80 is formed, which is a cut-out for regulating the position of aroma display 60 such that female screw part 76 faces the correct direction when aroma display 60 is mounted on booster 62. When a positioning member formed on booster 62 is fitted in positioning section 80, the position of aroma display 60 is regulated, and aroma display 60 is mounted on the correct position of booster 62.

On the second surface of side surface 82, a USB (Universal Serial Bus)-C connector 78 for aroma display 60 to receive control signals and power from outside is provided. Aroma display 60 contains six aroma cartridges, not shown, and has a function of emitting aroma or scent from the cartridge designated by the control signal from opening 64.

Referring in particular to FIG. 5, on the lower surface 72 of aroma display 60, a connector part 84 to the substrate 102 shown in FIG. 2 is formed. Of the control signals received through USB-C connector 78, the signal and power for controlling centrifugal fan 112 are applied to fan assembly 104 through connector part 84 and substrate 102, and used for driving centrifugal fan 112.

### C. Booster 62

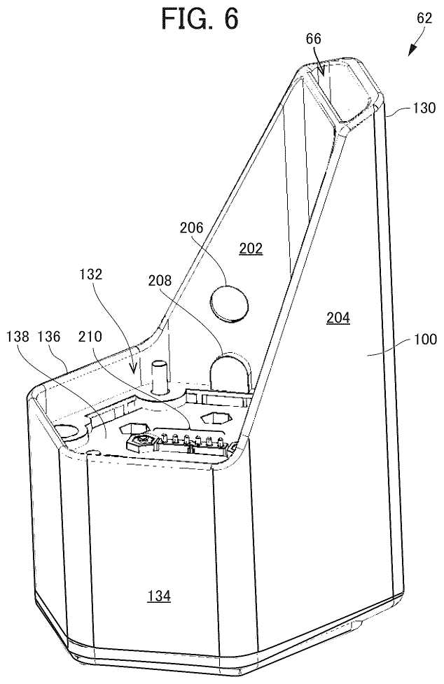

Referring to FIGS. 6 to 11, holder 100 of booster 62 includes: a display holder part 132 for holding the lower surface 72 and side surface 82 therearound of aroma display 60; and a fan housing 134 formed below and continuous from display holder part 132, for housing fan assembly 104.

Display holder part 132 is separated by partition plate 138 from fan housing 134. Of the outer circumference of partition plate 138, on three, mutually continuous sides, outer peripheral wall 136 is formed. Of the remaining three sides, on those sides adjacent to the sides where outer peripheral wall 136 is formed, sidewalls 202 and 204 are formed, respectively, which side walls are formed to extend obliquely upward from the outer peripheral wall 136. On the remaining one side sandwiched by sidewalls 202 and 204, duct 130 is provided, and an outer wall of duct 130 separates the inside of duct 130 from display holder part 132. Duct 130 has: an opening, not shown, formed at a portion to be in contact with the outer periphery of aroma display 60 at the lower surface side as the second surface of aroma display 60 when aroma display 60 is housed in display holder part 132; and an opening 66 provided in contact with the side connecting upper ends of sidewalls 202 and 204. The upper end of duct 130 is approximately of the same height as the upper ends of sidewalls 202 and 204. The opening 66 formed at the upper end of duct 130 comes to be positioned substantially flush with the upper surface, that is, the first surface, of aroma display 60 when the aroma display 60 is stored in the display holder part 132.

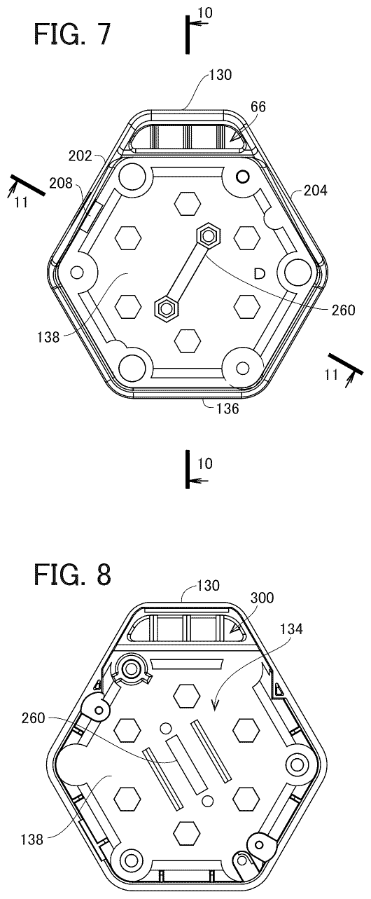

Referring to FIGS. 7 and 8, opening 66 formed at the upper end of duct 130, that is, formed on the upper surface side of aroma display 60, has an approximately trapezoidal cross-section. At the lower end of duct 130, that is, on the lower surface side of aroma display 60, a duct bottom opening 300 is formed, which has the same shape and same size (area) as opening 66. Duct bottom opening 300 is connected to deflector duct 114 shown in FIG. 2. As shown in FIG. 6, duct 130 is formed perpendicular to the bottom surface of booster 62, straight along the side surface. Therefore, the inner space of duct 130 is a prism having opening 66 and duct bottom opening 300 as opposite ends and approximately trapezoidal cross-section. Specifically, the cross-sectional shape and area of duct 130 in a plane perpendicular to the direction of extension of duct 130 are the same over the entire length of duct 130. By introducing air from centrifugal fan 112 to the opening of deflector duct 114 on the side opposite to the duct 130, the air is guided to the inside of duct 130.

At the central portion of partition plate 138, a pin-receiving opening 260 is formed, to which a pin 210 (see FIG. 6) of substrate 102 shown in FIG. 2 is inserted from the lower surface to the upper surface of partition plate 138. When this pin 210 is electrically coupled to connector part 84 shown in FIG. 5, the control signal from aroma display 60 is applied through substrate 102 to fan assembly 104.

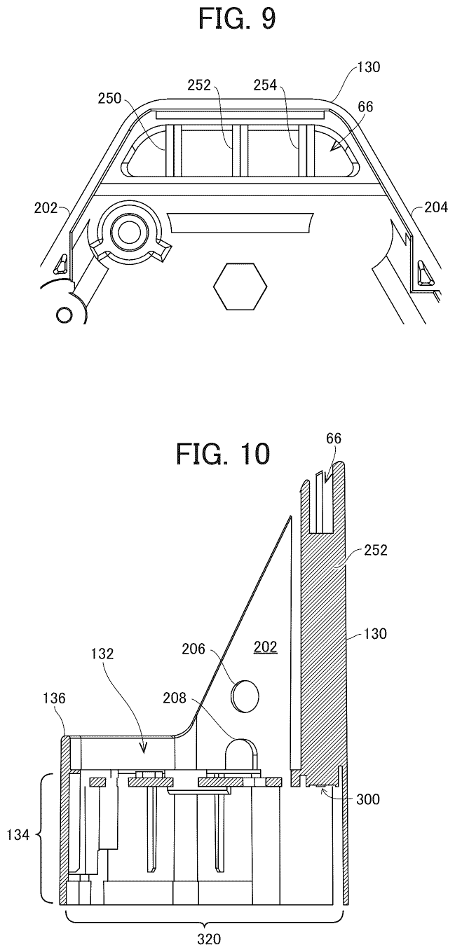

Referring to FIG. 9, in duct 130, three flow straighteners 250, 252 and 254 are provided, along the direction of air flow in duct 130. These flow straighteners are arranged spaced by a prescribed distance from and parallel to each other. Centrifugal fan 112 has a discharge outlet facing a direction different from the direction of extension of duct 130. In the present embodiment, the air flow fed from centrifugal fan 112 must be bent 90 degrees to the direction of duct 130. Further, in order to introduce the air flow fed from centrifugal fan 112 into duct 130, it is necessary not only to bend the air flow by 90 degrees but also to widen the air flow to the left and right directions. This causes disturbance of the air flow introduced to duct 130. By flow straighteners 250, 252 and 254 provided in duct 130, the air flow in the duct can be straightened and, as a result, scented air can be delivered far.

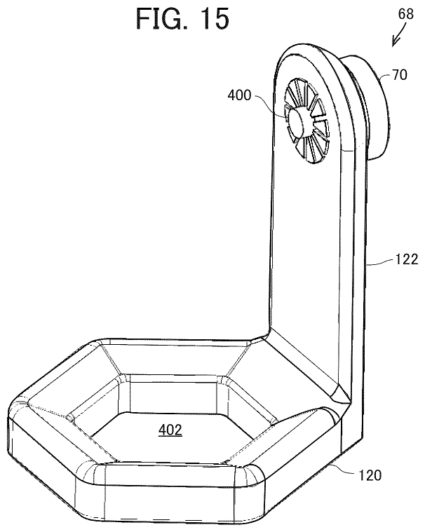

Referring to FIG. 10, on sidewall 202 of display holder part 132, a locking opening 206 is formed. Further, on the inner circumferential surface of sidewall 202 immediately therebelow, a positioning member 208 is formed, which engages with positioning section 80 (see FIGS. 4 and 5) provided in aroma display 60 to regulate the position of aroma display 60. When aroma display 60 is inserted to display holder part 132 such that positioning member 208 engages with positioning section 80, locking opening 206 faces the female screw part 76 shown in FIGS. 4 and 5. Therefore, it is possible to screw the male screw part 400 (see FIG. 15) of booster lock 70 of stand 68 through locking opening 206 into female screw part 76. As a result, aroma display 60 and booster 62 can be fixed on stand 68, with holder 100 held between aroma display 60 and booster lock 70.

A fan housing 134 is formed continuous from the lower part of outer peripheral wall 136, and it serves as a fan container 320 containing a fan (not shown) therein.

### D. Deflector Duct 114

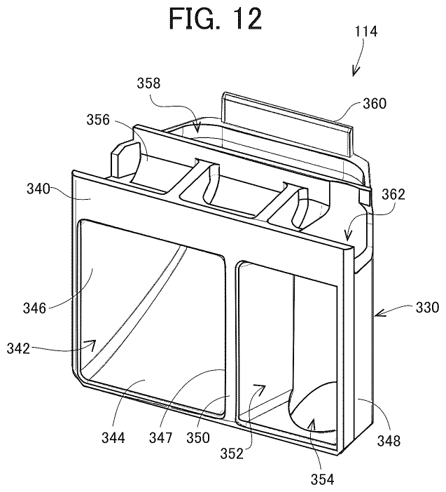

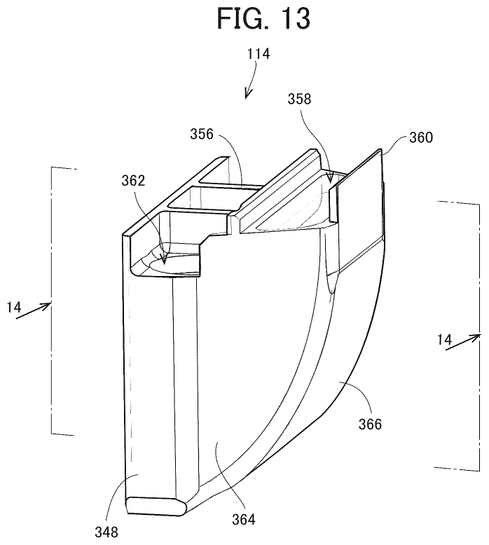

Referring to FIGS. 12 to 14, deflector duct 114 includes a housing 330 having: a front surface 340 on which a fan-side opening 342 connected to the air discharge outlet of centrifugal fan 112 (see FIG. 2) is formed; a sidewall 348; a rear surface 366; a side surface 364 connecting sidewall 348 and rear surface 366; and an upper partition 356. On front surface 340, a screw chamber 352 is formed, which is partitioned from fan-side opening 342 by a central partition 350. At the bottom surface of screw chamber 352 and at the upper partition 356, screw holes 354 and 362 are formed, respectively. These screw holes are to receive long screw 108 shown in FIG. 2.

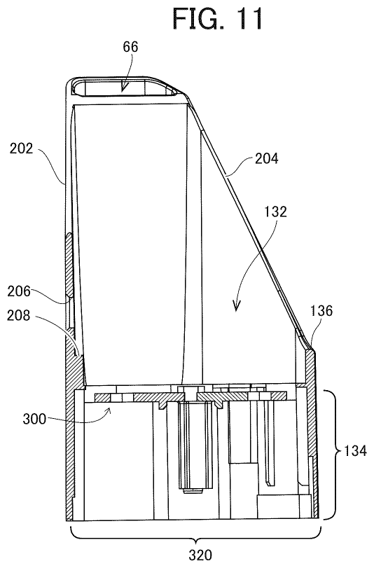

A duct-side opening 358 is formed at the upper surface of deflector duct 114, and the duct-side opening 358 is connected to duct bottom opening 300 of duct 130. Fan-side opening 342 and duct-side opening 358 are surrounded by bottom plate 344, sidewalls 346 and 347 and the lower surface of upper partition 356, which are parts of housing 330, thus forming an air passage that guides the air flow emitted by centrifugal fan 112 to the duct bottom opening 300 (see FIGS. 10 and 11). Around the duct-side opening 358, an engaging member 360 is formed. When engaging member 360 engages with duct bottom opening 300 of duct 130, deflector duct 114 comes to be tightly connected to duct bottom opening 300.

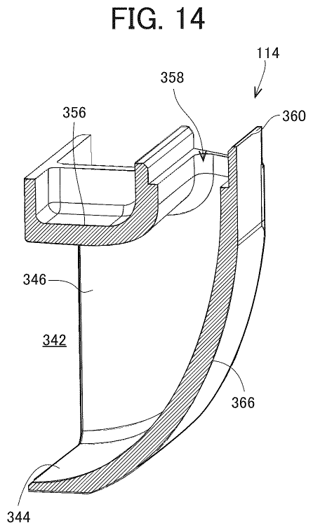

Particularly as shown in FIG. 14, bottom plate 344 and its rear surface 366 are streamlined with no step. Therefore, energy loss of the air flow emitted from centrifugal fan 112 can be minimized and the air flow can be guided to the inside of duct 130 with the air flow direction changed by 90 degrees. As a result, it is possible to deliver scented air far or to dissipate lingering scent, utilizing the strongest air wind and maximum static pressure of centrifugal fan 112.

### E. Stand 68

Referring to FIG. 15, stand 68 includes bottom part 120 and lock holder plate 122, as described above. The bottom part 120 has a shape of regular hexagon with an opening 402 inside. From the outer side of one of the six sides of regular hexagon of the bottom part 120, lock holder plate 122 is erected, extending perpendicular to the plane defined by the regular hexagon of the bottom part 120. Tip end of lock holder plate 122 is shaped semi-circular. Male screw part 400 of booster lock 70 is attached to lock holder plate 122 to pass through the center of semi-circle. Booster lock 70 is freely rotatable on lock holder plate 122. By screwing male screw part 400 of booster lock 70 into female screw part 76 of aroma display 60 provided in booster 62, it is possible to hold aroma display 60 and booster 62 on stand 68. By loosening booster lock 70 a little, it is possible to rotate aroma display 60 and booster 62 in a prescribed angular range with respect to stand 68, and by tightening booster lock 70 at the rotated position, aroma display 60 and booster 62 can be fixed at the rotated angle.

As shown in FIG. 2, on the rear surface of bottom part 120, six magnets 124 are embedded and fixed. Therefore, by attaching stand 68 on a part formed of steel, such as a cabinet door, the posture of aroma display 60 and booster 62 can be set freely.

### F. Operation

Referring to FIGS. 1 to 15, aroma display system 50 operates in the following manner. Referring to FIG. 1 in particular, in response to an external control signal, aroma display 60 emits air containing scent of an arbitrary combination of six different aromas from the opening 64, at timing designated by the control signal. The power of emission of aroma display 60 is relatively small. Therefore, the emitted scented air reaches only a small area around aroma display 60.

If it is desired to deliver the aroma to wider area, centrifugal fan 112 (see FIG. 2) of booster 62 is activated, timed with the emission of scented air by aroma display 60. The air emitted sideways from centrifugal fan 112 is changed to an upward flow by deflector duct 114, and guided to duct bottom opening 300 of duct 130. The air introduced to the inside of duct 130 through duct bottom opening 300 flows directly upward through duct 130 and blown out from the uppermost opening 66. The air flow generated by centrifugal fan 112 and blown out from duct 130 is fast and strong. Therefore, the air blown out from duct 130 draws in the surrounding air and reaches far. The scented air that had only reached the vicinity of aroma display 60 is drawn into the air blown out from duct 130 and carried farther away. As a result, the aroma emitted from aroma display 60 can be delivered farther than when booster 62 is not used.

Sometimes, aroma display 60 emits a certain aroma and then emits another aroma. In this situation, if the former aroma lingers, it is undesirably mixed with the latter aroma, and the desired effect cannot be attained. Booster 62 may also be activated in such a case. By activating booster 62, the air containing lingering aroma in the vicinity of aroma display 60 is blown out far away from aroma display 60. By blowing out a new scent from aroma display 60 after stopping booster 62, undesirable mixing of the new aroma with the lingering aroma can be prevented, and intended effect of the aroma can be realized.

When booster 62 is used, the air blown out from centrifugal fan 112 passes through deflector duct 114 and duct bottom opening 300 of duct 130 into duct 130, and emitted through opening 66. In the passage of air formed by deflector duct 114 and duct 130, there is nothing that hinders the air flow. Therefore, the air is blown out from opening 66 while the power applied to the air by centrifugal fan 112 is not lost. Therefore, it is possible to deliver the air emitted from aroma display 60 far enough or to dissipate scented air lingering around aroma display 60 immediately. As a result, even a relatively compact centrifugal fan 112 is sufficiently effective. It is unnecessary to increase the size of booster 62 and it can be compact. Further, operating noise of centrifugal fan 112 can be reduced.

As described above, according to the first embodiment, aroma display 60 is not placed in the air flow emitted from centrifugal fan 112, but the duct is provided along only a part of the outer wall of aroma display 60 and the air is emitted from the duct. When aroma display 60 emits scented air through the opening 64 formed at the central portion of aroma display 60, the scented air is delivered far on the air flow emitted from duct 130.

In the booster disclosed in Patent Literature 1, the air emitted from axial fan hits the bottom surface of aroma display and passes therearound to the discharge outlet of the booster. Therefore, it seems that by the booster disclosed in Patent Literature 1, the power of axial fun could not be fully utilized in the air flow.

In contrast, according to the present embodiment, the air emitted from the centrifugal fan is not hindered by aroma display 60 and is blown out smooth from duct 130. Therefore, the power of centrifugal fan can be fully utilized to generate the air flow and the maximum static pressure is attained. As a result, the scented air emitted from aroma display 60 can be drawn into the air flow efficiently and delivered farther. Further, the centrifugal fan is arranged in fan housing 134 on the bottom surface side of aroma display 60, and duct 130 is arranged only along a part of outer circumference of aroma display 60. As a result, the effect that the size of booster 62 is reduced can also be attained. As described above, as compared with the booster disclosed in Patent Literature 1, comparable or higher maximum static pressure can be attained using a smaller centrifugal fan. Thus, the effect of further reducing the size of booster 62 can be attained.

### 2. Second Embodiment

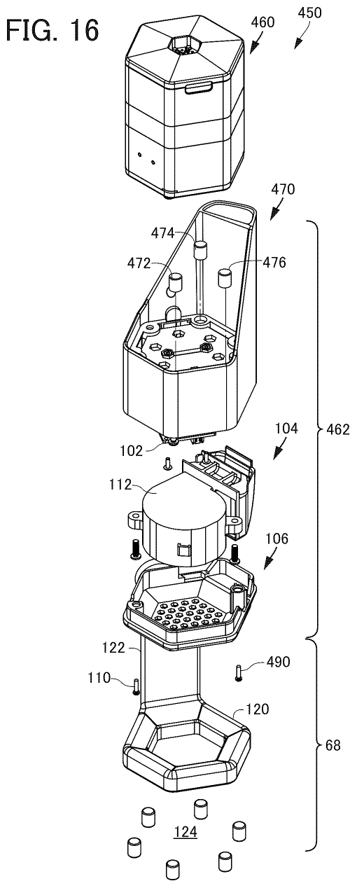

FIG. 16 shows a schematic structure of an aroma display system 450 in accordance with a second embodiment of the present invention. Referring to FIG. 16, the first difference between aroma display system 450 and aroma display system 50 of the first embodiment shown in FIG. 2 is that in place of aroma display 60, an aroma display 460 is used, to which iron or other metal that adheres to magnet is attached at an appropriate position on the bottom surface.

The second difference is that the system includes a booster 462 having, in place of holder 100, a holder 470 on which a plurality of magnets 472, 474 and 476 are provided for fixing aroma display 460 in display holder part 132. Therefore, different from the example of FIG. 2, it is unnecessary to fix aroma display 60 by means of long screw 108. In the second embodiment, long screw 108 shown in FIG. 2 is not used, and bottom plate 106 is fixed on the lower side of holder 470 by a small screw 490.

Except for these differences, aroma display system 450 has the same structure as aroma display system 50 in accordance with the first embodiment. The difference is only the manner how to fix the aroma display on the holder. Therefore, the second embodiment attains the same effects as the first embodiment.

### 3. Third Embodiment

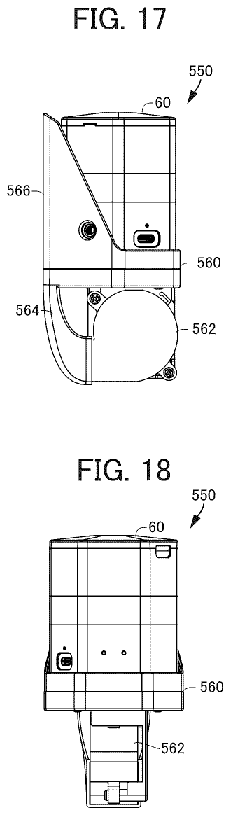

FIGS. 17 and 18 show front and side views of an aroma display system 550 in accordance with a third embodiment of the present invention. In FIGS. 17 and 18, stand 68 is not shown. As shown in FIG. 18, however, at a position of the holder of aroma display system 550 facing the female screw part of aroma display 60, an opening is formed. Therefore, aroma display system 550 can also be combined with stand 68.

The first difference between aroma display system 550 and aroma display system 50 in accordance with the first embodiment is that it includes a holder 560 not having a fan housing, in place of holder 100 of the first embodiment. This difference, however, is not essential, and fan housing 134 may be provided in this embodiment also. Holder 560 has a duct 566 extending from the bottom surface to the upper end of holder 560 along a side surface corresponding to one side of the outer circumference of hexagonal column of the holder 560, similar to duct 130 of the first embodiment. Though not shown, duct 566 has openings of the same shape at upper and lower ends thereof.

The second difference is that it includes, in place of centrifugal fan 112 of aroma display system 50, a centrifugal fan 562 arranged such that its rotation axis is in a direction perpendicularly intersecting the central axis of aroma display 60. By arranging centrifugal fan 562 in this manner, the discharge outlet of centrifugal fan 562 comes to face the direction perpendicular to the central axis of aroma display 60.

The third difference is that in place of deflector duct 114 of the first embodiment, a deflector duct 564 is provided, for guiding the air emitted from centrifugal fan 562 to the opening at the lower end of duct 566. The function of deflector duct 564 is the same as that of deflector duct 114.

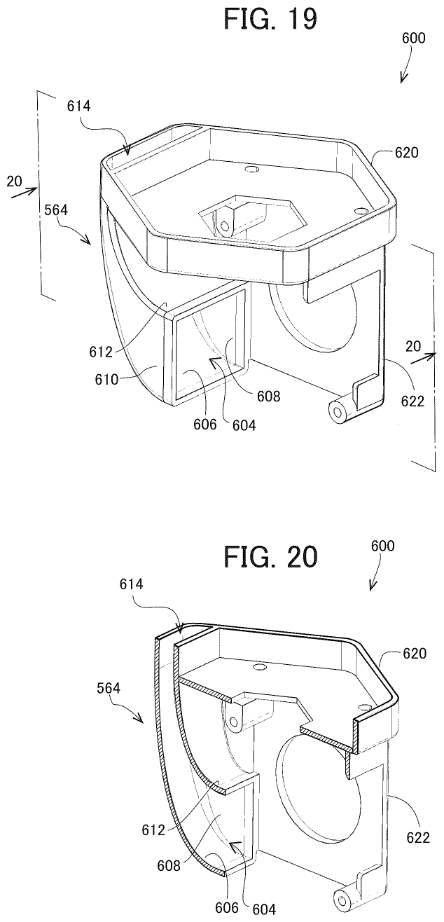

Referring to FIGS. 19 and 20, deflector duct 564 is a part of fan attachment member 600 that is integrally molded of resin. In addition to deflector duct 564, fan attachment member 600 includes: a holder receiving part 620 having a regular hexagonal circumference, connected to the bottom part of holder 560; and a fan attachment plate 622 mounted perpendicularly to the bottom part of holder receiving part 620. A screw hole is formed in fan attachment plate 622, and centrifugal fan 562 (see FIGS. 17 and 18) can be fixed by screw on fan attachment plate 622.

In the present embodiment, deflector duct 564 is formed along a part of fan attachment plate 622 at the bottom part of holder receiving part 620. Deflector duct 564 has: a duct-side opening 614 formed at a position flash with the upper end of holder receiving part 620; and a fan-side opening 604 formed to be connected to the air discharge outlet of centrifugal fan 562 fixed on fan attachment plate 622.

More specifically, deflector duct 564 includes: a sidewall 610, a sidewall 608 formed along fan attachment plate 622, a bottom surface 606 and an upper surface 612. Sidewalls 610 and 608, bottom surface 606 and the fan-side end of upper surface 612 define the circumference of fan-side opening 604. On the other hand, circumference of duct-side opening 614 is defined by the upper end of bottom surface 606, upper ends of sidewalls 610 and 608 and one side of hexagon of holder receiving part 620.

As described above, the function of deflector duct 564 is the same as deflector duct 114 of the first embodiment. Bottom surface 606 and upper surface 612 form a streamlined air flow passage. Therefore, deflector duct 564 of the present embodiment also minimizes energy loss of the air flow emitted from centrifugal fan 562, changes the direction of air flow by 90 degrees and guides the air flow to the inside of duct 130. As a result, it is possible to deliver the scented air far away or to dissipate lingering scent, utilizing the maximum wind and maximum static pressure of centrifugal fan 562.

On the other hand, deflector duct 564 differs significantly from deflector duct 114 in its shape.

By way of example, when a sirocco fan is used as the axial fan, its discharge outlet has a rectangular shape. The longer side of rectangle is substantially the same as the longitudinal size of the impeller blade. The shorter side of rectangle is substantially the same as the widthwise size of the impeller. Generally, the longitudinal size of an impeller is larger than the widthwise size.

When centrifugal fan 112 is arranged as centrifugal fan 112 of FIG. 2, the longer side of discharging outlet is the same as the vertical (up-down) direction of FIG. 2. Specifically, in the first embodiment, air flow that is long in the vertical direction and short in the horizontal direction is fed from centrifugal fan 112. In order to guide this air flow to the inside of duct 130, it is necessary to change the direction of air flow from lateral direction to longitudinal direction. Further, it is necessary to change the lateral air flow that is distributed short in the horizontal direction and long in the vertical direction to the longitudinal air flow fitting the inner shape of duct 130. When the air flow distributed long in the vertical direction is changed in this manner, the air flow is inevitably disturbed. Further, the disturbance of air flow still remains when the air is discharged from duct 130. Therefore, the air blown out from duct 130 proceeds with some breadth.

On the other hand, when centrifugal fan 562 is arranged as shown in FIGS. 17 and 18, the length of longer side of discharge outlet of centrifugal fan 562 can be made substantially the same as the width of the inside of duct 566. Therefore, deflector duct 564 simply needs to change the direction of air flow laterally discharged from centrifugal fan 562 by 90 degrees to upward, and adjustment of two-dimensional distribution of the air flow is unnecessary. Further, as described above, the inner shape of deflector duct 564 is smooth, and there is no factor that hinders the air flow. As a result, in the third embodiment, the air flow discharged from duct 566 is hardly disturbed and, therefore, aroma can be delivered farther.

It is noted, however, that in the third embodiment, since the air flow is hardly disturbed, the effect of expanding aroma is weaker than the first embodiment. Therefore, the arrangement of the third embodiment is preferable when pinpoint delivery of aroma to a certain location is desired. On the other hand, by the arrangement of the first embodiment, while such a pinpoint-delivery is difficult, the aroma is delivered with some breadth and, therefore, the aroma can efficiently be delivered not much affected by the user's position.

In the embodiments above, the inner circumference of booster 62 is hexagonal. This is simply because the inner circumference of booster 62 is formed to match the outer circumference of aroma display 60, and the inner circumference of booster 62 is not limited to a hexagon. It also applies to the outer circumference of booster 62, which is also hexagonal.

Further, in the above-described embodiments, duct 130 is formed along only one side of the hexagon of outer circumference of booster 62. The present invention, however, is not limited to such an embodiment. For example, the duct may be formed along two or three sides of the hexagon. Considering the object of making the booster small, the upper limit is provision of the duct along three sides of the hexagon. If the duct is formed along four or more sides, the booster will be large. Further, since the duct comes to have a larger cross-sectional area, it becomes difficult to attain sufficient static pressure unless the fan size is also increased.

The duct may be provided to extend over a part of a side and a part of adjacent side of the hexagon with a vertex in-between, of outer circumference of booster 62. Similarly, the duct may be provided extending over one side and a part of its left side and a part of its right side.

Further, the outer circumference of booster 62 is not limited to a hexagon. The outer circumference of booster 62 is made hexagonal simply because the outer circumference of aroma display 60 is a hexagon. For example, if the outer circumference of aroma display 60 is octagonal, it is natural to make the outer circumference of booster 62 octagonal. Naturally, the outer circumference of aroma display 60 may be a polygon other than a regular polygon, or it may be a circle or any other shape. In that case, it is natural that the outer circumference of booster 62 is made to match the outer circumferential shape of aroma display 60. When the outer circumference of booster is made circular, the width of booster duct cannot be defined by the number of sides, different from the case of a polygon. In that case, it is preferable to provide the duct in the range of 1/10 to ⅓, preferably, from ⅛ to ¼ and, more preferably, from 1/7 to ⅕ of the outer circumference of the booster. Here, the inner cross-sectional area of duct may not be the substantial trapezoid as in the embodiment above, and the duct may be deformed to have a curve along the outer circumference of the booster.

The embodiments as have been described here are mere examples and should not be interpreted as restrictive. The scope of the present invention is determined by each of the claims with appropriate consideration of the written description of the embodiments and embraces modifications within the meaning of, and equivalence to, the languages in the claims.

### REFERENCE SIGNS LIST

50, 450, 550 aroma display system
60,460 aroma display
62, 462 booster
68 stand
70 booster lock
80 positioning section
86 central axis
100, 470, 560 holder
104 fan assembly
108 long screw
112, 562 centrifugal fan
114, 564 deflector duct
130, 566 duct
132 display holder part
134 fan housing
136 outer peripheral wall
138 partition plate
202, 204, 346, 347, 348, 608, 610 sidewall
206 locking opening
208 positioning member
250, 252, 254 flow straightener
300 duct bottom opening
320 fan container
342, 604 fan-side opening
358, 614 duct-side opening

1. For an aroma display having a first surface, a second surface opposite to the first surface, and a third surface connecting outer circumferences of the first and second surfaces and discharging aroma from said first surface, a booster device for enhancing aroma emission power, comprising:
a holder part holding said aroma display;
a duct having first and second openings, provided along said third surface; and
a centrifugal fan for feeding air to said first opening; wherein
said duct is provided on said holder part such that said first opening is positioned on the side of said second surface of said aroma display held by said holder part and said second opening is positioned on the side of said first surface of said aroma display held by said holder part;
said duct is provided such that the central axis of the duct is apart from the central axis of said aroma display by a prescribed distance and is parallel to the central axis of said aroma display;
outer circumference of said aroma display has a shape of a polygon; and
said duct is formed to be adjacent only to at most two continuous sides of said polygon.

2. The booster device according to claim 1, further comprising
a stand for swingably supporting said aroma display and said booster device by engaging with an engaging section formed on said third surface of said aroma display through an opening formed in a side surface portion of said holder part.

3. The booster device according to claim 1, wherein said duct has the same cross-sectional area and the same cross-sectional shape from said first opening to said second opening.
